(19) United States
(12) Patent Application Publication
(10) Pub. No.: US 2024/0009263 A1
(43) Pub. Date: Jan. 11, 2024

(54) COMPOUNDS FOR TREATING ENVELOPED VIRUS INFECTIONS

(71) Applicant: Progelife, Marseille (FR)

(72) Inventors: Eric DESSAUD, Marseille (FR); Pierre CAU, Aix en Provence (FR)

(21) Appl. No.: 18/254,327

(22) PCT Filed: Nov. 24, 2021
(86) PCT No.: PCT/EP2021/082875

§ 371 Date: May 24, 2023

(30) Foreign Application Priority Data
Nov. 24, 2020 (EP) 20306432.4

(51) Int. Cl. A61K 38/05; A61P 31/14; A61K 38/06

(52) U.S. Cl. CPC A61K 38/05; A61P 31/14; A61K 38/06

(57) ABSTRACT

The present invention relates to a compound of the following formula (I):

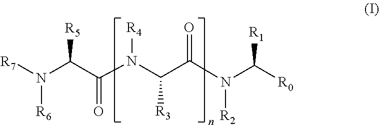

or a pharmaceutically acceptable salt thereof,
for use in a method for preventing or treating an infection by an enveloped virus in an individual.

### FIELD OF THE INVENTION

The present invention relates to compounds for treating infections by enveloped viruses, in particular belonging to the Coronaviridae family, more particularly by the virus SARS-CoV-2.

### TECHNICAL BACKGROUND

In December 2019 an outbreak of pneumonia cases of unknown origin occurred in Wuhan in China and spread quickly nationwide. On Jan. 7, 2020, the causative pathogen was identified as a novel coronavirus, which was named 2019-nCoV and later SARS-CoV-2.

The new virus is closely related to both SARS-CoV (82% nucleotide identity) and MERS-CoV (50% nucleotide identity), yet distinct from them.

Early mortality rates suggested that COVID-19, the name for the disease caused by SARS-CoV-2, may be less severe than SARS and MERS. However, illness onset among rapidly increasing numbers of people rapidly suggested that SARS-CoV-2 would be more contagious than both SARS-CoV and MERS-CoV. As of Oct. 28, 2020, about 44 million cases of COVID-19 (in accordance with the applied case definitions and testing strategies in the affected countries) have been reported, including 1 166 923 deaths.

Besides the race for coronavirus vaccines (DeFrancesco (2020). Nat Biotechnol 38(10): 1132-1145), a great deal of effort has been made and is still ongoing to find effective drugs against the new and unknown SARS-CoV-2.

Among the various compounds tested, some of them (e.g. remdesivir, a drug previously developed for the treatment of Ebola virus infections, anti-malarial chloroquine and hydroxychloroquine) have been reported to show promising efficacy and acceptable safety in treating cultured Vero cells infected by SARS-CoV-2. However, these drugs have no proven efficiency in human patients.

Only corticosteroïds are associated with reduced mortality in critically ill patients with COVID-19 (Prescott and Rice (2020). JAMA 324: 1292-1295).

Accordingly, there is still a need for alternative treatments of infections by SARS-CoV-2.

### SUMMARY OF THE INVENTION

The present invention arises from the unexpected finding, by the inventors, that certain peptidic compounds could be effective for treating infection by enveloped virus, in particular by SARS-CoV-2.

Accordingly, the present invention relates to a compound of the following formula (I):

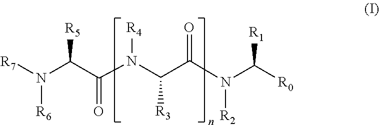

wherein:
n represents: 0, 1 or 2;
R₀ represents an aldehyde group or a protected aldehyde group;
R₂, R₄, and R₆, identical or different, with the proviso that when n=2 the two R₄ groups may be identical or different, represent: H (hydrogen atom); an alkyl group having from 1 to 6 carbon atoms, optionally substituted by one or more amino groups or carboxylic acid groups; or an alkaryl or aryl group having from 5 to 10 carbon atoms, optionally substituted by one or more amino groups, hydroxyl groups or carboxylic acid groups; in particular an isobutyl group, an isopentyl group, a phenylethyl group or an hydroxyphenylethyl group;
R₁, R₃ and R₅, identical or different, with the proviso that when n=2 the two R₃ groups may be identical or different, represent: H (hydrogen atom), an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group; and
R₇ represents:
   a protecting group, or
   a group of the following formula (II):

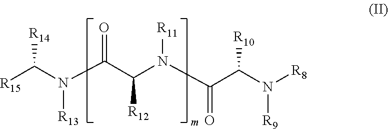

wherein:
   m represents: 0, 1 or 2;
   R₉, R₁₁ and R₁₃, identical or different, with the proviso that when n=2 the two R₁₁ groups may be identical or different, represent: H (hydrogen atom); an alkyl group having from 1 to 6 carbon atoms, optionally substituted by one or more amino group or carboxylic acid group; or an alkaryl or aryl group having from 5 to 10 carbon atoms, optionally substituted by one or more amino groups, hydroxyl groups, or carboxylic acid groups; in particular an isobutyl group, an isopentyl group, a phenylethyl group or an hydroxyphenylethyl group;
   R₁₀, R₁₂ and R₁₄, identical or different, with the proviso that when n=2 the two R₁₂ groups may be identical or different, represent: H (hydrogen atom), an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group;
   R₈ represents a linking moiety; and
   R₁₅ represents an aldehyde group or a protected aldehyde group
      or a pharmaceutically acceptable salt thereof,
      for use as a medicament or in a method for preventing or treating an infection by an enveloped virus, in particular of the Coronaviridae family, in an individual.

The present invention also relates to a compound of formula (I) as defined above or a pharmaceutically acceptable salt thereof for use as defined above, in combination with at least one other compound suitable for the prevention or treatment of an infection by an enveloped virus, in particular of the Coronaviridae family, more particularly by SARS-CoV-2.

The present invention also relates to a method for the prevention or treatment of an infection by an enveloped virus, in particular of the Coronaviridae family, in an individual, comprising administering to the individual an effective amount of a compound of formula (I) as defined above or a pharmaceutically acceptable salt, ester, hydrate, derivative, prodrug or metabolite thereof.

The present invention also relates to a method as defined above, wherein the compound of formula (I) as defined above or a pharmaceutically acceptable salt thereof is administered in combination with at least one other compound suitable for the prevention or treatment of an infection by an enveloped virus, in particular of the Coronaviridae family, more particularly by SARS-CoV-2.

The present invention also relates to a pharmaceutical composition, comprising as active substance a compound of formula (I) as defined above or a pharmaceutically acceptable salt thereof in particular for use in the prevention or treatment of an infection by an enveloped virus, in particular of the Coronaviridae family, in an individual.

The present invention also relates to a pharmaceutical composition for use as defined above further comprising at least one other compound suitable for the prevention or treatment of an infection by an enveloped virus in particular of the Coronaviridae family, more particularly by SARS-CoV-2.

The present invention also relates to products containing:
a compound of formula (I) as defined above or a pharmaceutically acceptable salt thereof, and
at least one other compound suitable for the prevention or treatment of an infection by an enveloped virus, in particular of the Coronaviridae family, more particularly by SARS-CoV-2,
   as a combined preparation for simultaneous, separate or sequential use in the prevention or treatment an infection by an enveloped virus in an individual.

### DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Compound

Compounds of formula (I) can be readily synthesized by one of skill in the art, in particular by using solid phase peptide synthesis (SPPS).

Protecting groups for protecting the N-terminus of peptides are well known to one of skill in the art and any such protecting group can be used according to the invention. However, it is preferred that the protecting group according to the invention is selected from the group consisting of carboxybenzyl (Z or Cbz), acetyl (Ac), dichlorobenzyl, pyrazinyl carbonyl, difluorophényl.

As intended herein, an aldehyde group is a group of the following formula:

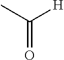

Protected aldehyde groups are well known to one of skill in the art and any such protected aldehyde group can be used according to the invention. However, it is preferred that the protected aldehyde group according to the invention is selected from the group consisting of an amide, a carboxylic acid, a semicarbazone, an imine, an oxyme, an hydrazone, a sodium bisulfite and a thiazolidine. More preferably, the protected aldehyde group according to the invention is selected from the groups represented by the following formulae:

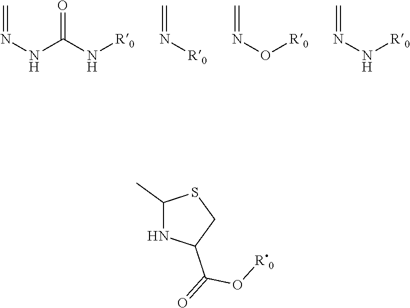

wherein R′₀ represents H (hydrogen atom) or a group comprising from 1 to 100 carbon atoms, preferably from 1 to 50 carbon atoms and more preferably from 1 to 20 carbon atoms. Where R′₀ represents a group comprising from 1 to 20, 50 or 100 carbon atoms, it is preferably a polar group or a polymer ligation group. Most preferably, the protected aldehyde group according to the invention is represented by the following formula:

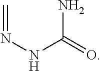

As intended herein, a linking moiety refers to any group capable of bridging two amine groups. Preferably, the linking moiety has from 3 to 20 carbon atoms and comprises at least 2 carboxylic acid groups. As will clear to one of skill in the art the two carboxylic acid groups of the preferred liking moiety form amide bonds with the two amine groups the linking moiety is bridging. More preferably, the linking moiety, when bridging the two amine groups is selected from the groups having the following formulae:

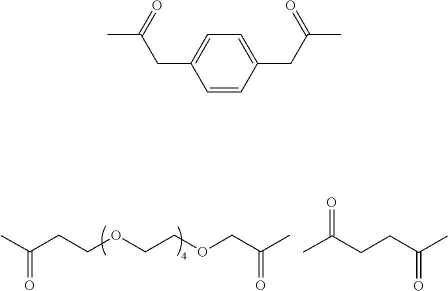

As should be clear to one of skill in the art, when R₇ represents a group of formula (II), the compound of formula (I) can be represented by the following formula (III):

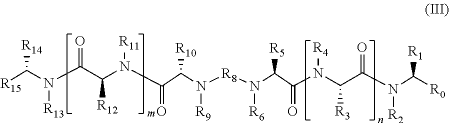

In a preferred embodiment of the compound of formula (I) according to the invention, R₅ is not a pyrazinyl protecting group.

In another preferred embodiment of the compound of formula (I) according to the invention, R₀ is not a boronic group.

In another preferred embodiment of the compound of formula (I) according to the invention, n=1, R₂ is H (hydrogen atom), R₇ is a protecting group selected from acetyl (Ac), pyrazinyl, difluorophenyl and carboxybenzyl (Z), R₁, R₃ and R₅, identical or different, represent a Leucine (Leu, L) functional group, a Norvaline (Nva) functional group, or a Phenylalanine (Phe, F) functional group.

In another preferred embodiment of the compound of formula (I) according to the invention, the protecting group is acetyl (Ac), pyrazinyl, difluorophenyl or carboxybenzyl (Z), provided that when the protecting group is Z R₁, R₃ and R₅ do not all represent a Leucine (Leu, L) functional group.

In another preferred embodiment of the compound of formula (I) according to the invention, the protecting group is acetyl (Ac) or carboxybenzyl (Z), provided that when the protecting group is Z, R₁, R₃ and R₅ do not all represent a Leucine (Leu, L) functional group.

In another preferred embodiment of the compound of formula (I) according to the invention:
R₇ is a carboxybenzyl (Z), R₅ and R₃ represent a Leucine (Leu, L) functional group, and R₁ represents a Norvaline (Nva) functional group;
R₇ is acetyl (Ac), and R₁, R₃ and R₅ represent a Leucine (Leu, L) functional group;
R₇ is carboxybenzyl (Z), R₅ represents a Phenylalanine (Phe, F) functional group, and R₁ and R₃ represent a Leucine (Leu, L) functional group;
R₇ is carboxybenzyl (Z), R₅ and R₃ represents a Leucine (Leu, L) functional group, and R₁ represent a Phenylalanine (Phe, F) functional group;
R₇ is carboxybenzyl (Z), R₅ represents a Leucine (Leu, L) functional group, R₃ represents a Phenylalanine (Phe, F) functional group, and R₁ represents a Leucine (Leu, L) functional group;
R₇ is pyrazinyl, and R₁, R₃ and R₅ represent a Leucine (Leu, L) functional group;
R₇ is difluorophenyl, and R₁, R₃ and R₅ represent a Leucine (Leu, L) functional group.

Preferably, the compound of formula (I) according to the invention is selected from the group consisting of:

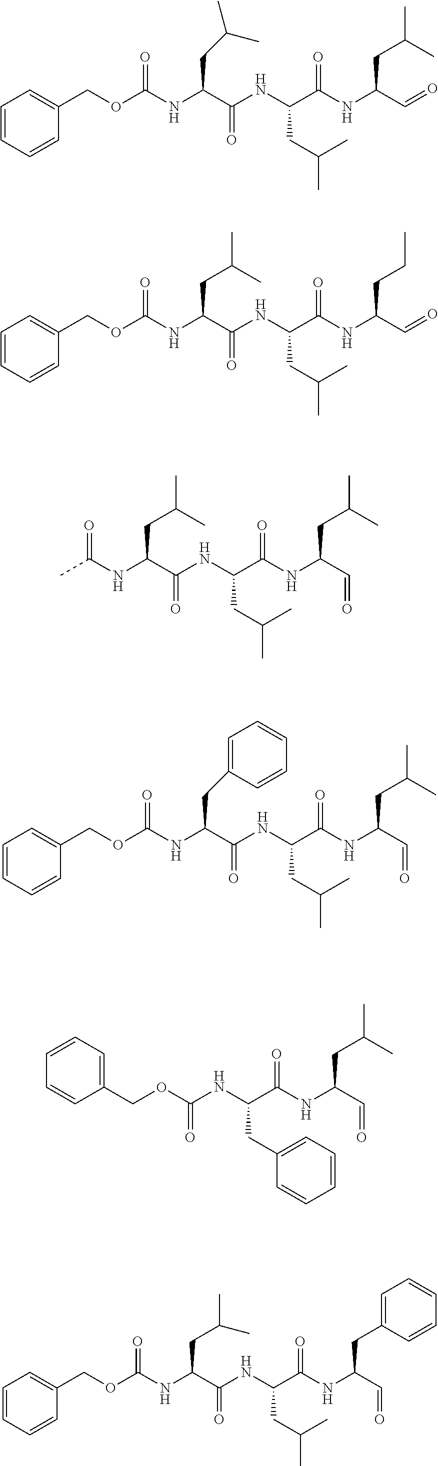

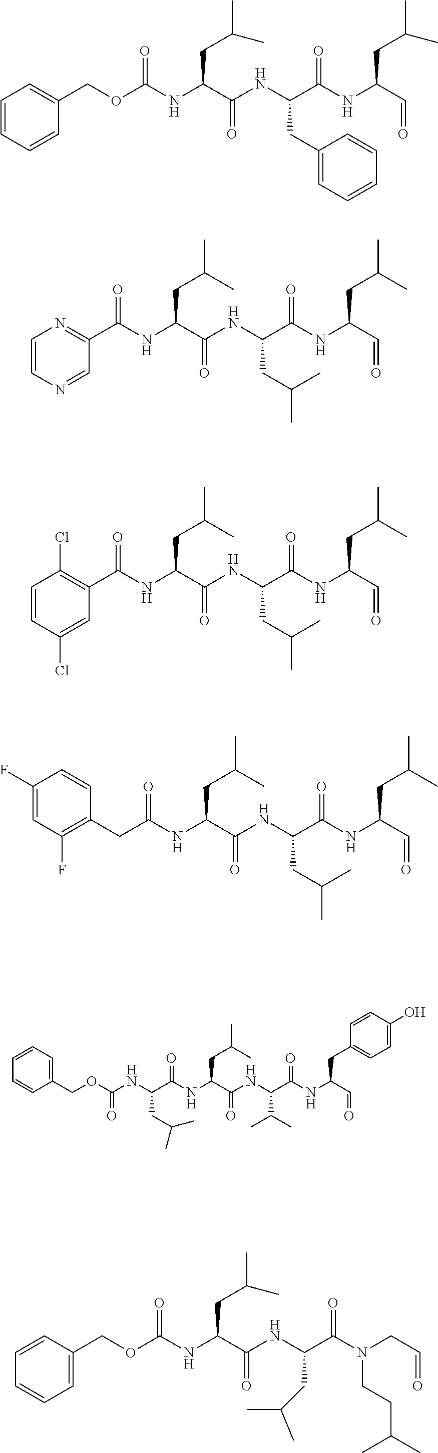 [SMILES: CC(C)C[C@@H](C=O)NC(=O)[C@H](Cc1ccccc1)NC(=O)[C@H](CC(C)C)NC(=O)OCc1ccccc1 ; CC(C)C[C@@H](C=O)NC(=O)[C@H](CC(C)C)NC(=O)[C@H](CC(C)C)NC(=O)c1cnccn1 ; CC(C)C[C@@H](C=O)NC(=O)[C@H](CC(C)C)NC(=O)[C@H](CC(C)C)NC(=O)c1cc(Cl)ccc1Cl ; CC(C)C[C@@H](C=O)NC(=O)[C@H](CC(C)C)NC(=O)[C@H](CC(C)C)NC(=O)Cc1ccc(F)cc1F ; CC(C)C[C@H](NC(=O)OCc1ccccc1)C(=O)N[C@@H](CC(C)C)C(=O)N[C@H](C(=O)N[C@H](C=O)Cc1ccc(O)cc1)C(C)C ; CC(C)CCN(CC=O)C(=O)[C@H](CC(C)C)NC(=O)[C@H](CC(C)C)NC(=O)OCc1ccccc1]

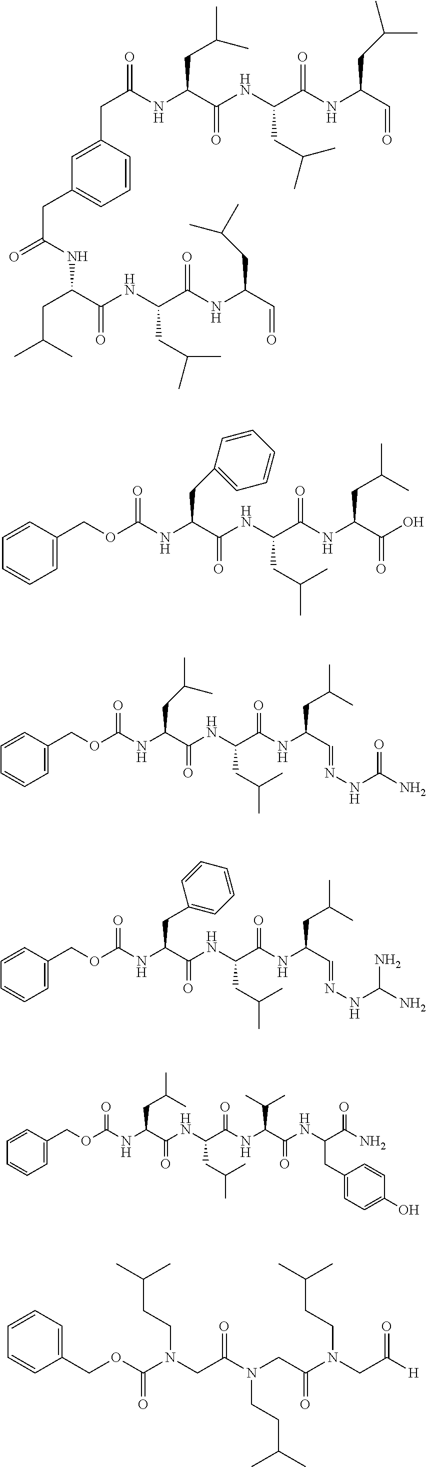

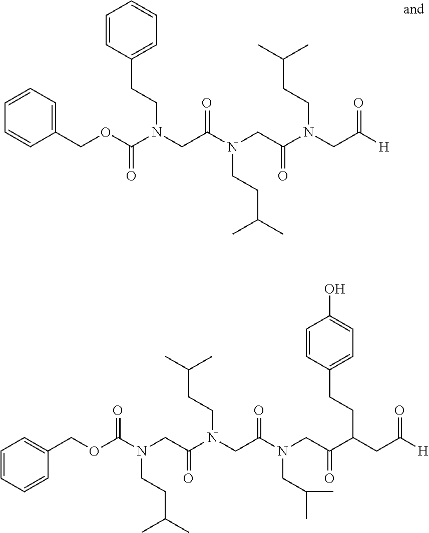 [SMILES: CC(C)CCN(CC=O)C(=O)CN(CCC(C)C)C(=O)CN(CCc1ccccc1)C(=O)OCc1ccccc1 ; CC(C)CCN(CC(=O)N(CC(=O)C(CC=O)CCc1ccc(O)cc1)CC(C)C)C(=O)CN(CCC(C)C)C(=O)OCc1ccccc1]

More preferably, the compound of formula (I) according to the invention is selected from the group consisting of:

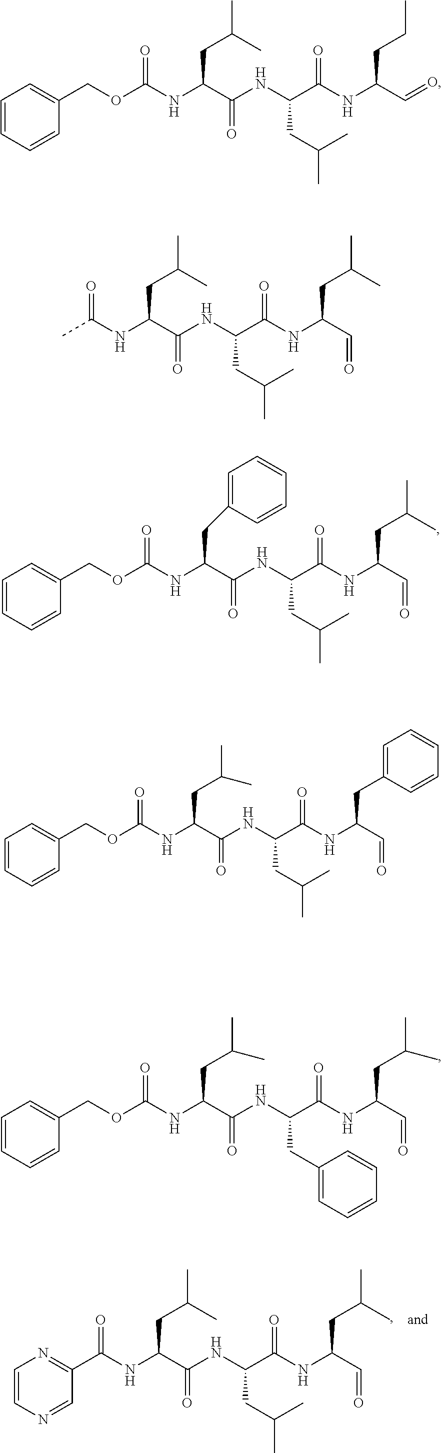

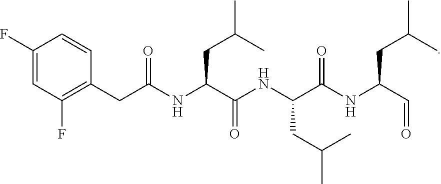 [SMILES: CC(C)C[C@@H](C=O)NC(=O)[C@H](CC(C)C)NC(=O)[C@H](CC(C)C)NC(=O)Cc1ccc(F)cc1F]

In a preferred embodiment of the invention, the compound of formula (I) according to the invention is different from:

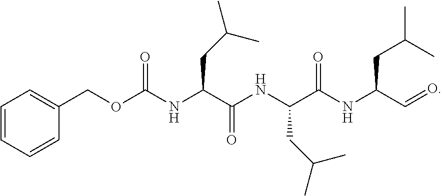 [SMILES: CC(C)C[C@@H](C=O)NC(=O)[C@H](CC(C)C)NC(=O)[C@H](CC(C)C)NC(=O)OCc1ccccc1]

### Virus

As intended herein, an enveloped virus is a virus that has an outer wrapping or envelope. This envelope comes from the infected cell, or host, in a process called “budding off.” During the budding process, newly formed virus particles become “enveloped” or wrapped in an outer phospholipidic coat that is made from a small piece of the cell's plasma membrane.

Preferably, the virus as defined above is:
an Herpesviridae virus, in particular Herpes simplex virus, varicella-zoster virus, cytomegalovirus, Epstein-Barr virus,
a Pleolipoviridae virus, in particular HHPV1, HRPV1, HGPV1, His2V,
a Togaviridae virus, in particular Rubella virus, alphavirus,
an Arenaviridae virus, in particular Lymphocytic choriomeningitis virus,
a Flaviviridae virus, in particular Dengue virus, hepatitis C virus (HCV), yellow fever virus, Zika virus,
an Orthomyxoviridae virus, in particular Influenzavirus A, influenzavirus B, influenzavirus C, isavirus, thogotovirus,
a Paramyxoviridae, in particular Measles virus, mumps virus, respiratory syncytial virus, Rinderpest virus, canine distemper virus,
a Bunyaviridae virus, in particular California encephalitis virus, hantavirus,
a Rhabdoviridae virus, in particular Rabies virus,
a Filoviridae virus, in particular Ebola virus, Marburg virus,
a Coronaviridae, in particular Coronavirus,
a Bornaviridae virus, in particular Borna disease virus,
an Arteriviridae virus, in particular Arterivirus, equine arteritis virus,
a Retroviridae virus, in particular HIV, more particularly HIV-1 or HIV-2,
an Hepadnaviridae virus, in particular hepatitis B virus (HBV).

Preferably, the enveloped virus is of the Coronaviridae family.

Preferably, the virus as defined above is of the Alphacoronavirus, Betacoronavirus, Deltacoronavirus, or Gammacoronavirus genus, more preferably of the Betacoronavirus genus, most preferably of the Sarbecovirus or the Merbecovirus sub-genus.

Preferably also the virus as defined above is a human virus, i.e. a virus which can infect a human.

Preferably, the virus as defined above is selected from the group consisting of SARS-CoV, SARS-CoV-2, MERS-CoV and mutants or variants thereof.

Preferably, the virus as defined above is SARS-CoV-2, or a mutant or variant thereof.

SARS-CoV-2 is notably described in Fuk-Woo Chan et al. (2020) Emerging Microbes & Infections 9:221-236, which is incorporated herein by reference, and is also named 2019-nCoV, HCoV-19, SARS2, COVID-19 virus, Wuhan coronavirus, Wuhan seafood market pneumonia virus, and Human coronavirus 2019.

Preferably, the virus as defined above is SARS-CoV-2 and has the genomic sequence defined by NCBI Reference Sequence NC_045512.2 (SEQ ID NO: 1), or the complementary thereof, or is a mutant or variant thereof.

As intended herein, a “mutant or variant” of a virus as defined above, or of a genomic sequence of a virus as defined above, has a genomic sequence or is a nucleotide sequence which has at least 85%, 90%, 95%, 96% 97%, 98%, 99% or 99.5% identity with the genomic sequence of the virus as defined above.

Mutant or variants of SEQ ID NO: 1 can in particular be found on the “NCBI virus” website by searching for SARS-CoV-2 taxid:2697049. A preferred variant of SARS-CoV-2 according to the invention harbours at least one mutation, in particular of the spike protein, selected from the group consisting of K417N, K417T, L452R, T478K, E484K, E484Q, N501 Y and D614G, which harbours an A-to-G nucleotide mutation at position 23403 in SEQ ID NO: 1. A preferred variant of SARS-CoV-2 according to the invention is a variant of concern, more preferably selected from the group consisting in B.1.1.7, B.1.1.7+E484K, B.1.351, P.1, B.1.617.1, B.1.617.2 and B.1.617.3

As intended herein, a first nucleotide sequence “having at least X % identity” with a second nucleotide sequence, in particular differs from the second sequence by the insertion, the suppression or the substitution of at least one nucleotide. Besides, the percentage of identity between two nucleotide sequences is defined herein as the number of positions for which the bases are identical when the two sequences are optimally aligned, divided by the total number of bases of the longer of the two sequences. Two sequences are said to be optimally aligned when the percentage of identity is maximal. Besides, as will be clear to one of skill in the art, it may be necessary to add gaps in order to obtain an optimal alignment between the two sequences. In addition, when calculating the percentage of identity between an RNA nucleotide sequence and a DNA nucleotide sequence, an Uracile (U) base and a Thymine (T) base at the same position are considered to be identical.

As intended herein preventing or treating an infection by a enveloped virus, in particular of the Coronaviridae family, in an individual, encompasses preventing or treating the symptoms, disorders, syndromes, conditions or diseases, such as pneumonia or COVID-19, associated to the infection by the enveloped virus, in particular of the Coronaviridae family, more particularly by SARS-CoV-2.

In particular, the present invention aims at preventing or treating long COVID, which is also known as post-COVID-19 syndrome, post-acute sequelae of COVID-19 (PASC), chronic COVID syndrome (CCS) and long-haul COVID. It is a condition characterized by long-term sequelae—appearing or persisting after the typical convalescence period—of coronavirus disease 2019 (COVID-19).

### Individual

Preferably, the individual is a bird, such as a chicken, or a mammal, such as a human, a canine, in particular a dog, a feline, in particular a cat, an equine, a bovine, a porcine, a caprine, such a sheep or a goat, or a camelidae, more preferably the individual is a human.

Preferably, the individual as defined above is a human. Preferably, the individual as defined above is a human aged 40 or more, more preferably 50 or more, more preferably 60 or more, even more preferably 70 or more and most preferably 80 or more.

Preferably, the individual as defined above is a male individual.

Preferably, the individual as defined above suffers from at least one other disease or condition, in particular selected from hypertension, diabetes, in particular type 2 diabetes, metabolic syndrome, a cardiovascular disease, in particular ischemic cardiomyopathy, a chronic respiratory disease, an auto-immune disease or cancer.

Preferably, the individual as defined above is overweight or obese.

According to a usual definition a human individual is considered overweight if its Body Mass Index (BMI, body weight in kg relative to the square of the height in meters) is higher than or equal to 25 kg/m² and less than 30 kg/m² and the individual will be said obese if his BMI is higher than or equal to 30 kg/m². The individual according to the invention may notably present with severe obesity, in particular characterized in human by a BMI higher than or equal to 35 kg/m².

More generally, it is preferred that the individual as defined above is a human and has a BMI higher than or equal to 25 kg/m², 26 kg/m², 27 kg/m², 28 kg/m², 29 kg/m², 30 kg/m², 31 kg/m², 32 kg/m², 33 kg/m², 34 kg/m², 35 kg/m² or 40 kg/m².

Besides, the individual as defined above may also have an abdominal obesity, corresponding in particular to a visceral adipose tissue excess. According to a usual definition, a male human individual has an abdominal obesity if the abdominal perimeter is higher than or equal to 94 cm, in particular higher than 102 cm and a female human individual has an abdominal obesity if the abdominal perimeter is higher than or equal to 80 cm, in particular higher than 88 cm. The abdominal perimeter measure is well known to one of skilled in the art: abdomen circumference is thus preferably measured midway between the last floating rib and the top of the iliac crest in a standing individual in gentle expiration.

It is particularly preferred that the individual as defined above is a man and presents with an abdominal perimeter higher than or equal to 90 cm, 91 cm, 92 cm, 93 cm, 94 cm, 95 cm, 96 cm, 97 cm, 98 cm, 99 cm, 100 cm, 101 cm or 102 cm. It is also preferred that the individual according to the invention is a woman and presents with an abdominal perimeter higher than or equal to 75 cm, 76 cm, 77 cm, 78 cm, 79 cm, 80 cm, 81 cm, 82 cm, 83 cm, 84 cm, 85 cm, 86 cm, 87 cm or 88 cm.

Preferably, the individual according to the invention is afflicted with COVID-19 or is at risk of being afflicted with COVID-19.

### Additional Compound

Preferably, the other compound suitable for the prevention or treatment of an infection by a virus of the Coronaviridae family, in particular by SARS-CoV-2, is selected from the group consisting of soluble recombinant human ACE, an anti-SARS-CoV-2 antibody, in particular a recombinant antibody; an anticoagulant, an aminobisphosphonate, apilimod, arbidol, azithromycin, bevacizumab, bromhexin hydrochloride, camostat mesylate, carfilzomib, carrimycin, chloroquine, chlorpromazine, cobicistat, danoprevir, darunavir, dexamethasone, eculizumab, favipiravir, fingolimod, hydroxychloroquine, interferon omega or its pegylated derivative, recombinant human interferon alpha 1A or its pegylated derivative, recombinant human interferon alpha 1B or its pegylated derivative, recombinant human interferon alpha 2A or its pegylated derivative, recombinant human interferon alpha 2B or its pegylated derivative, recombinant human interferon beta 1A or its pegylated derivative, recombinant human interferon beta 1B or its pegylated derivative, intravenous immunoglobulins, ledipasvir, lopinavir/ritonavir, lopinavir, ritonavir meplazumab, methylprednisolone, N-acetylcysteine, nafamostat, nicotine, nitazoxanide, oseltamivir, penciclovir, pirfenidone, remdesivir, ribavirin, sarilumab, sofobusvir, a statin, thalidomide, tocilizumab, velpadasvir, vitamin C, voxilaprevir, zinc, GS-441524, MK-711, molnupiravir, PF-07321332, PAXLOVID™ (PF-07321332+ritonavir) and pharmaceutically acceptable salts, esters, hydrates, derivatives, prodrugs or metabolites thereof.

### Pharmaceutical Composition

The compound of formula (I) according to the invention or a pharmaceutically acceptable salt thereof, optionally combined with at least one other compound suitable for the prevention or treatment of an infection by an enveloped virus, in particular of the Coronaviridae family, more particularly by SARS-CoV-2, can be comprised in a pharmaceutical composition which can comprise at least one pharmaceutically acceptable vehicle or excipient. The pharmaceutically acceptable vehicle or excipient can be selected from dispersants, solubilizers, nebulizers, stabilizers, preservatives, etc. Besides, pharmaceutically acceptable vehicle or excipient which can be used in formulations, in particular liquid and/or injectable formulations, are preferably selected from sucrose, lactose, starch, methylcellulose, hydroxymethylcellulose, carboxymethylcellulose, croscarmellose sodium, lactose monohydrate, magnesium stearate, microcrystalline cellulose, povidone, sodium lauryl sulfate, mannitol, gelatin, lactose, vegetable oils, acacia gum, liposomes, etc.

### Administration

As intended herein “combined” or “in combination” means that the compound of formula (I), or the pharmaceutical acceptable salt thereof, as defined above, is administered at the same time than the additional compound as defined above, either together, i.e. at the same administration site, or separately, or at different times, provided that the time period during which the composition as defined above exerts its pharmacological effects on the individual and the time period during which the additional compound exerts its pharmacological effects on the individual, at least partially intersect. Preferably, the compound of formula (I), or the pharmaceutical acceptable salt thereof, and the additional compound with which it is combined, exert a supra-additive or synergic effect in the prevention or treatment of an infection by an enveloped virus.

The compound of formula (I) or a pharmaceutically acceptable salt thereof or the pharmaceutical composition as defined above can be administered orally, parenterally, mucosally or cutaneously. The parenteral route preferably comprises subcutaneous, intravenous, intramuscular or intraperitoneal administration, although the latter is rather reserved for animals. The mucosal route preferably comprises nasal administration, oro-pharyngeal administration, pulmonary administration or administration via the rectal mucosa. The cutaneous route advantageously comprises the dermal route, in particular via a transdermal device, typically a patch.

The compound of formula (I) according to the invention or a pharmaceutically acceptable salt thereof or the pharmaceutical composition as defined above can be formulated in the form of injectable solutions or suspensions, gels, oils, tablets, suppositories, powders, gel capsules, capsules, aerosols, etc., optionally by means of administration forms or of devices which provide sustained and/or delayed release. For this type of formulation, an agent such as cellulose, carbonates or starches is advantageously used.

The compound of formula (I) according to the invention or a pharmaceutically acceptable salt thereof or the pharmaceutical composition as defined above can be administered to the individual as defined above at a dose between 0.1 mg and 1000 mg, preferably between 0.1 mg and 100 mg, more preferably between 1 mg and 100 mg, of the compound of formula (I) or a pharmaceutically acceptable salt thereof as defined above. Of course, those skilled in the art are able to adjust the dose of the compound of formula (I) or a pharmaceutically acceptable salt thereof as defined above according to the weight or the body surface area of the individual to be treated. Preferably, the dosage range of the compound of formula (I) according to the invention or a pharmaceutically acceptable salt thereof is from 0.1 mg/day and 1000 mg/day, preferably between 0.1 mg/day and 100 mg/day, more preferably between 1 mg/day and 100 mg/day.

The invention will be further specified by the following non-limiting Example.

### EXAMPLE

The antiviral properties of the compounds according to the invention were assessed according to the general method disclosed in Touret et al. (2020) “In vitro screening of a FDA approved chemical library reveals potential inhibitors of SARS-CoV-2 replication” Scientific Reports 10, 13093

### Material and Methods

### 1. Compounds

The following compounds were tested:

| Formula | Name |
|---|---|
| 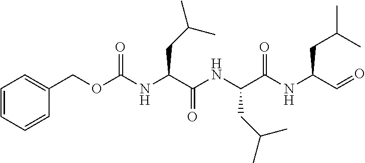 [SMILES: CC(C)C[C@@H](C=O)NC(=O)[C@H](CC(C)C)NC(=O)[C@H](CC(C)C)NC(=O)OCc1ccccc1] | 1 |
| 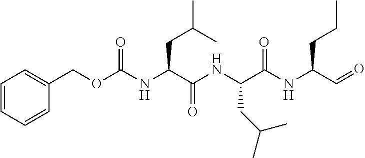 [SMILES: CCC[C@@H](C=O)NC(=O)[C@H](CC(C)C)NC(=O)[C@H](CC(C)C)NC(=O)OCc1ccccc1] | 2 |
| 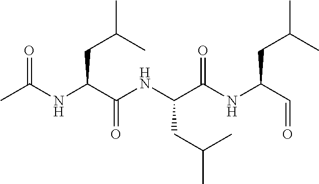 [SMILES: CC(=O)N[C@@H](CC(C)C)C(=O)N[C@@H](CC(C)C)C(=O)N[C@H](C=O)CC(C)C] | 3 |
| 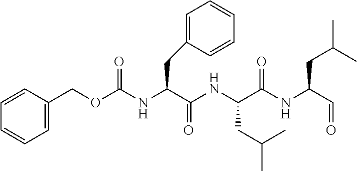 [SMILES: CC(C)C[C@@H](C=O)NC(=O)[C@H](CC(C)C)NC(=O)[C@H](Cc1ccccc1)NC(=O)OCc1ccccc1] | 4 |
| 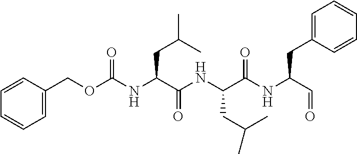 [SMILES: CC(C)C[C@H](NC(=O)OCc1ccccc1)C(=O)N[C@@H](CC(C)C)C(=O)N[C@H](C=O)Cc1ccccc1] | 5 |
| 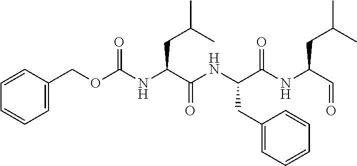 [SMILES: CC(C)C[C@@H](C=O)NC(=O)[C@H](Cc1ccccc1)NC(=O)[C@H](CC(C)C)NC(=O)OCc1ccccc1] | 6 |
| 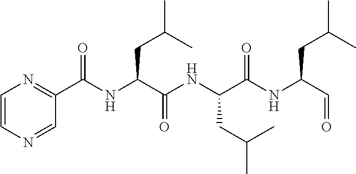 [SMILES: CC(C)C[C@@H](C=O)NC(=O)[C@H](CC(C)C)NC(=O)[C@H](CC(C)C)NC(=O)c1cnccn1] | 7 |
| 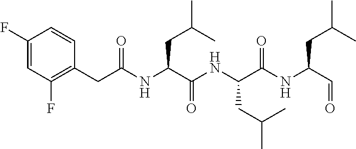 [SMILES: CC(C)C[C@@H](C=O)NC(=O)[C@H](CC(C)C)NC(=O)[C@H](CC(C)C)NC(=O)Cc1ccc(F)cc1F] | 8 |
| 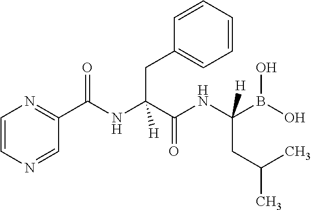 [SMILES: CC(C)C[C@H](NC(=O)[C@H](Cc1ccccc1)NC(=O)c1cnccn1)B(O)O] | 9 |

Compounds 1-8 are exemplary compounds according to the invention. Compound 9 is a comparative exemplary compound.

Remdesivir (Rem) was also tested as a positive control.

### 2. Cell Lines

VeroE6 (ATCC CRL-1586), Caco-2 (ATCC HTB-37) and VeroE6-TMPRSS2+ (NIBSC 100978) cells were grown in minimal essential medium (MEM; Thermofisher Scientific, Waltham, USA) with 5% heat-inactivated fetal calf serum (FCS; Thermofisher Scientific, Waltham, USA), at 37° C. with 5% CO2 with 1% penicillin/streptomycin (5000 U/mL and 5000 μg/mL respectively; Thermofisher Scientific, Waltham, USA) and supplemented with 1% non-essential amino acids (Thermofisher Scientific, Waltham, USA) and L-Glutamine (Thermofisher Scientific, Waltham, USA).

### 3. Viruses

SARS-CoV-2 strain BavPat1 was obtained from EVA GLOBAL. To prepare the virus working stock, a 25 cm2 culture flask of confluent VeroE6 cells growing with MEM medium with 2.5% FBS (Thermofisher Scientific, Waltham, USA) was inoculated at multiplicity of infection (MOI) of 0.001. Cell supernatant medium was harvested at the peak of replication and supplemented with 25 mM HEPES (Sigma, St Louis, USA) before being stored frozen in small aliquots at −80° C. Experiments were performed in biosafety level 3 facilities.

### 4. EC50 and CC50 Determination

One day prior to infection, 5×10⁴ VeroE6/Caco-2/VeroE6-TMPRSS2+ cells were seeded in 100 μL assay medium (containing 2.5% FCS) in 96 well plates. The next day, seven twofold serial dilutions of compounds (0.6-40 μM, in triplicate) were added to the cells (25 μL/well, in assay medium). Four virus control wells were supplemented with 25 μL of assay medium. After 15 min, 25 μL of a virus mix diluted in medium was added to the wells. The amount of virus working stock used was calibrated prior to the assay, based on a replication kinetics, so that the replication growth is still in the exponential growth phase for the readout. Four cell control wells (i.e. with no virus) were supplemented with 50 μL of assay medium. On each plate, a control compound (Remdesivir, BLDPHARM, Shanghai, China) was added in duplicate with seven twofold serial dilutions (0.16-20 μM, in duplicate). Plates were incubated for 2 days at 37° C. prior to quantification of the viral genome by real-time RT-PCR. To do so, 100 μL of viral supernatant was collected in S-Block (QIAGEN, Hilden, Germany) previously loaded with VXL lysis buffer containing proteinase K and RNA carrier. RNA extraction was performed using the Qiacube HT automat and the Cador Pathogen 96 HT kit following manufacturer instruction. Viral RNA was quantified by real-time RT-qPCR (EXPRESS One-Step Superscript qRT-PCR Kit, universal Invitrogen using 3.5 μL of RNA and 6.5 μL of RT qPCR mix and standard fast cycling parameters, i.e., 10 min at 50° C., 2 min at 95° C., and 40 amplification cycles (95° C. for 3 s followed by 30 s at 60° C.). Quantification was provided by four 2 log serial dilutions of an appropriate T7-generated synthetic RNA standard of known quantities (10² to 10⁸ copies). RT-qPCR reactions were performed on QuantStudio 12K Flex Real-Time PCR System (APPLIED BIOSYSTEMS, Waltham, USA) and analyzed using QuantStudio 12 K Flex Applied Biosystems software v1.2.3. Primers and probe sequences, which target SARS-CoV-2N gene, were: Fw: GGC CGC AAA TTG CAC AAT (SEQID NO: 2); Rev: CCA ATG CGC GAC ATTCC (SEQ ID NO: 3); Probe: FAM-CCC CCA GCG CTT CAG CGT TCT-BHQ1 (SEQ ID NO: 4). The 50% and 90% effective concentrations (EC50, EC90; compound concentration required to inhibit viral RNA replication by 50% and 90%) were determined using logarithmic interpolation. For the evaluation of the CC50 (the concentration that reduces the total cell number by 50%), the same culture conditions were set as for the determination of the EC50, without addition of the virus, and cell viability was measured using CellTiter Blue (PROMEGA, Fitchburg, USA). CC50 was determined using logarithmic interpolation. For each tested drug, EC50 and EC90 were determined and the relative effectiveness in inhibiting viral replication compared to inducing cell death is defined as the therapeutic or selectivity index (TI/SI=CC50 value/EC50 value). Compounds with a high TI/SI ratio are sought.

### Results

The results obtained on Vero-E6 cells are shown in the following table:

| μM | 1 | 2 | 3 | 4 | 5 | 6 | 7 | 8 | 9 | Rem |
|---|---|---|---|---|---|---|---|---|---|---|
| EC50 | 0.12 | 0.06 | <0.3 | 0.13 | 0.06 | 0.31 | 0.68 | 0.52 | 1.05 | 2.18 |
| EC90 | 0.5 < EC90 < 1 | 0.16 | 1.57 | 0.60 | 1.93 | 11.20 | 1.93 | 1.28 | 21.53 | 2.45 |
| CC50 | 2.79 | >2 | 13.56 | 6.46 | >2 | 1.68 | >2 | >2 | 0.23 | |
| SI = CC50/EC50 | 22.8 | >33.3 | >45.2 | 49.69 | >33.3 | 12.92 | >2.94 | >3.84 | 0.22 | |

Compounds 2, 3, 4, 5 and 6 present the best selectivity index (SI), i.e. the best balance between virus inhibition and cytotoxicity.

Compounds 1, 7 and 8 also present an advantageous balance, although less well characterized than that of compounds 2, 3, 4, 5 and 6.

The balance between virus inhibition and cytotoxicity of comparative compound 9 is not favourable.

The EC50 of Remdesivir (Rem) (positive control) is higher than that of the compounds according to the invention, i.e. it is less potent.

Three compounds have been assessed on Caco-2 cells and results are summarized in the table below:

| μM | 1 | 2 | 5 |
|---|---|---|---|
| EC50 | 0.12 | 0.13 | 0.09 |
| EC90 | 0.13 | 0.63 | 0.47 |
| CC50 | 0.23 | 0.45 | 0.48 |
| SI = CC50/EC50 | 1.89 | 3.50 | 5.13 |

Finally, results have been obtained with four compounds tested on VeroE6-TMPRSS2+ cells in a pilot study and results are presented in the table below:

| μM | 1 | 4 | 5 | 6 | Rem |
|---|---|---|---|---|---|
| EC50 | 0.85 | >2 | 1.78 | 0.14 | 1.32 |
| EC90 | 3.30 | >2 | 2.30 | >2 | 1.66 |

1. A method for preventing or treating an infection by an enveloped virus in an individual, comprising administering a compound of formula (I): 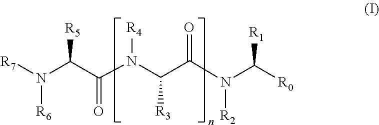
wherein:
n represents: 0, 1 or 2;
R₀ represents an aldehyde group or a protected aldehyde group;
R₂, R₄, and R₆, identical or different, with the proviso that when n=2 the two R₄ groups may be identical or different, represent: H; an alkyl group having from 1 to 6 carbon atoms, optionally substituted by one or more amino groups or carboxylic acid groups; or an alkaryl or aryl group having from 5 to 10 carbon atoms, optionally substituted by one or more amino groups, hydroxyl groups or carboxylic acid groups;
R₁, R₃ and R₅, identical or different, with the proviso that when n=2 the two R₃ groups may be identical or different, represent: H, an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group; and
R₇ represents:
a protecting group, or
a group of formula (II): 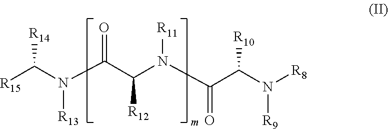
wherein:
m represents: 0, 1 or 2;
R₉, R₁₁ and R₁₃, identical or different, with the proviso that when n=2 the two R₁₁ groups may be identical or different, represent: H; an alkyl group having from 1 to 6 carbon atoms, optionally substituted by one or more amino group or carboxylic acid group; or an alkaryl or aryl group having from 5 to 10 carbon atoms, optionally substituted by one or more amino groups, hydroxyl groups or carboxylic acid groups;
R₁₀, R₁₂ and R₁₄, identical or different, with the proviso that when n=2 the two R₁₂ groups may be identical or different, represent: H, an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group;
R₈ represents a linking moiety; and
R₁₅ represents an aldehyde group or a protected aldehyde group;
or a pharmaceutically acceptable salt thereof, to the individual.

2. The method according to claim 1, wherein the virus is of the Coronaviridae family.

3. The method according to claim 1, wherein the virus is SARS-CoV, SARS-CoV-2, MERS-CoV, or mutants or variants thereof.

4. The method according to claim 1, wherein the virus is SARS-CoV-2, or a mutant or variant thereof.

5. The method according to claim 1, wherein the individual is aged 40 or more.

6. The method according to claim 1, wherein the individual suffers from at least one other disease or condition.

7. The method according to claim 1, further comprising administering at least one other compound suitable for the prevention or treatment of an infection by an enveloped virus.

8. The method according to claim 1, wherein n=1, R₂ is H, R₇ is a protecting group selected from acetyl (Ac), pyrazinyl, difluorophenyl and carboxybenzyl (Z), R₁, R₃ and R₅, identical or different, represent a Leucine (Leu, L) functional group, a Norvaline (Nva) functional group, or a Phenylalanine (Phe, F) functional group.

9. The method according to claim 8, wherein the protecting group is acetyl (Ac), pyrazinyl, difluorophenyl, or carboxybenzyl (Z), provided that when the protecting group is Z, R₁, R₃ and R₅ do not all represent a Leucine (Leu, L) functional group.

10. The method according to claim 8, wherein:
R₇ is a carboxybenzyl (Z), R₅ and R₃ represent a Leucine (Leu, L) functional group, and R₁ represents a Norvaline (Nva) functional group;
R₇ is acetyl (Ac), and R₁, R₃ and R₅ represent a Leucine (Leu, L) functional group;
R₇ is carboxybenzyl (Z), R₅ represents a Phenylalanine (Phe, F) functional group, and R₁ and R₃ represent a Leucine (Leu, L) functional group;
R₇ is carboxybenzyl (Z), R₅ and R₃ represents a Leucine (Leu, L) functional group, and R₁ represent a Phenylalanine (Phe, F) functional group;
R₇ is carboxybenzyl (Z), R₅ represents a Leucine (Leu, L) functional group, R₃ represents a Phenylalanine (Phe, F) functional group, and R₁ represents a Leucine (Leu, L) functional group;
R₇ is pyrazinyl, and R₁, R₃ and R₅ represent a Leucine (Leu, L) functional group; or
R₇ is difluorophenyl, and R₁, R₃ and R₅ represent a Leucine (Leu, L) functional group.

11. A method for preventing or treating an infection by an enveloped virus in an individual, comprising administering a pharmaceutical composition comprising as active ingredient a compound of formula (I) as defined in claim 1, or a pharmaceutically acceptable salt thereof, optionally in association with at least one pharmaceutically acceptable carrier or excipient, to the individual.

12. The method according to claim 11, further comprising administering at least one other compound suitable for the prevention or treatment of an infection by an enveloped virus.

13. A method for preventing or treating an infection by an enveloped virus in an individual, comprising administering a pharmaceutical composition comprising as active ingredients:
a compound of formula (I) according to claim 1, or a pharmaceutically acceptable salt thereof, and
at least one other compound suitable for the prevention or treatment of an infection by an enveloped virus,
optionally in association with at least one pharmaceutically acceptable carrier or excipient.

14. A method for preventing or treating an infection by an enveloped virus in an individual, comprising administering simultaneously, separately, or sequentially;
a compound of formula (I) according to claim 1, or a pharmaceutically acceptable salt thereof, and
at least one other compound suitable for the prevention or treatment of an infection by an enveloped virus, to the individual.

15. The method according to claim 1, wherein the virus is of the Betacoronavirus genus.

16. The method according to claim 6, wherein at least one other disease or condition is hypertension, diabetes, a cardiovascular disease, a chronic respiratory disease, or cancer.


SEQUENCE LISTING (4 sequences)

SEQ ID NO: 1 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 18 bp, Artificial
  PCR primer
ggccgcaaattgcacaat

SEQ ID NO: 3 - DNA, 17 bp, Artificial
  PCR primer
ccaatgcgcgacattcc

SEQ ID NO: 4 - DNA, 21 bp, Artificial
  Probe
cccccagcgcttcagcgttct